(19) United States
(12) Patent Application Publication
(10) Pub. No.: US 2010/0291010 A1
(43) Pub. Date: Nov. 18, 2010

(54) NOVEL ESTERS AND COMPOSITIONS AND USES THEREOF

(75) Inventors: Samad Syed, Paramus, NJ (US); Phillip Cotrell, Salisbury, NC (US)

Correspondence Address: BURNS & LEVINSON, LLP, 125 SUMMER STREET, BOSTON, MA, 02110, US

(73) Assignee: INNOSPEC LIMITED, ELLESMERE PORT, CHESHIRE (GB)

(21) Appl. No.: 12/810,218

(22) PCT Filed: Dec. 23, 2008
(86) PCT No.: PCT/GB08/51227

§ 371 Date: Aug. 5, 2010

(30) Foreign Application Priority Data
Dec. 28, 2007 (GB) 0725266.1

(51) Int. Cl. A61K 8/37; C07C 69/78; A61Q 17/04; A61Q 15/00; A61Q 19/00

(52) U.S. Cl. 424/60; 560/103; 424/65; 514/544

(57) ABSTRACT

A novel ester of an optionally substituted aromatic acid (for example benzoic acid) and a branched C₁₃ primary alcohol is suitable for use in providing improved cosmetic and personal care compositions including sunscreen, antiperspirant and deodorant compositions.

This invention relates to aromatic esters (for example benzoate esters) of C13 branched chain fatty alcohols, and their use in topical cosmetic compositions.

U.S. Pat. No. 4,275,222 describes benzoic acid esters of a mixture of C₁₂, C₁₃, C₁₄ and C₁₅ primary alcohols, and their use as diluents, solvents, plasticizers and liquid carriers, notably for use in toiletry or cosmetic products, dyestuffs or textiles. It is stated that in such alcohols at least 70% by weight of the alcohol of each specific chain length is linear. The branching (if any) comprises about 50% of methyl groups with smaller amounts of ethyl, propyl, butyl, aryl and hexyl groups. The alcohols are said to be commercially available.

It has now been determined that certain esters which may be present as components within the mixtures described in U.S. Pat. No. 4,275,222, when added as an emollient to a cosmetic composition, particularly a sunscreen composition, provides new and unexpected properties when compared to said mixtures.

In accordance with a first aspect of this invention there is provided an ester of an optionally substituted aromatic acid and a branched C₁₃ primary alcohol.

A preferred ester is an ester of an optionally substituted benzoic acid and a branched C₁₃ primary alcohol. A general formula for said preferred ester may be given as

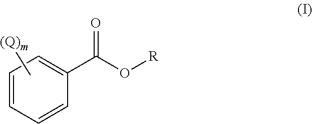

where Q is a substituent, as later defined; m is 0 (zero) or an integer from 1 to 5 (the groups Q being the same as or different from each other when m is 2, 3, 4 or 5); and R is a branched C₁₃ alkyl group.

The benzene ring (or other aromatic ring system) may be substituted with 0 to 5 groups (in addition to the reacted acid group COOH). For example it may be tri- or di-substituted in the ring (or ring system) or, more preferably, mono-substituted. Substitution may be at the ortho, and/or meta, and/or para position(s).

The or each substituent may be any common group used for substitution of a benzene ring (or other aromatic ring system), for example one or more of an alkyl group (an especially preferred substituent), an alkenyl group, a haloalkyl group, an alkynyl group, a nitrile group, a carboxylic acid (in addition to the reacted carboxylic acid group), an ester, an ether, an alkoxy group, a haloalkoxy group, a halo group, a hydroxyl group, a mercapto group, an alkylmercapto group, an alkylsulfoxy group, a sulfoxy group, an aryl group, an arylalkyl group, a substituted or unsubstituted amine group or a nitro group. Within these substituents an alkyl group, including of the alkyl moiety of an alkyl-containing group (for example alkoxy or haloalkyl) is suitably a C₁₋₄ alkyl group, more preferably a methyl group. Alternatively, and most preferably, the benzene ring (or other aromatic ring system) is unsubstituted (i.e. m is zero).

The branched C₁₃ primary alcohol backbone may be branched at one or more positions of the backbone of the primary alcohol.

The branched C₁₃ primary alcohol residue is preferably branched from one position only, of the backbone of the primary alcohol.

The branched C₁₃ primary alcohol residue preferably contains from 1 to 6 carbon atoms, in one or more side chains.

The branched C₁₃ primary alcohol residue preferably has a C₁₋₆ alkyl side chain. A C₃₋₆ alkyl side chain, when present, is preferably an n-C₃₋₆ alkyl group. However a preferred side chain is an ethyl group or, especially, a methyl group.

Preferably the branched C₁₃ primary alcohol residue is branched from the carbon atom second furthest from the optionally substituted aromatic moiety.

An especially preferred ester of the first aspect of the present invention has the formula

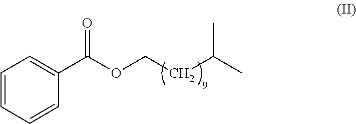

Preferably a compound of the first aspect is provided in at least 90% by weight pure form, more preferably at least 95%, and most preferably at least 98%. In an especially preferred embodiment it is provided in a substantially pure form.

It has been discovered that esters of the first aspect, for example compounds of formula I or II, can produce enhanced properties when added to cosmetic compositions. These enhanced properties make such compounds beneficial for use in cosmetic products such as skin care and personal care products. These products may be in the form of a cream, lotion, bar or stick. The compounds of formula I or II have new and unexpected properties as cosmetic additives not only as an emollient but also as solvents and liquid carriers for a cosmetically active material in the cosmetic preparation.

Esters of the first aspect may be made by analogous esterification reactions to those used for the preparation of other fatty alcohol-aromatic acid esters.

In accordance with a preferred embodiment of this invention, the ester is suitably free of other fatty alcohol or fatty acid esters; and is suitably in a substantially pure state.

In accordance with a second aspect of the present invention there is provided a composition for application to the person, for example to hair, skin or nails, the composition comprising as one component an ester of the first aspect. Said composition may hereinafter be called a cosmetic composition or a topical composition or a skin benefit composition.

Further components of the composition may comprise a carrier or diluent or solvent; and/or a cosmetically effective ingredient. Such further components may be as hereinafter described.

Preferably the composition of the second aspect constitutes at least 40% wt, preferably at least 50% wt, more preferably at least 70% wt, most preferably at least 90% wt, of the total weight of C₁₂-C₁₅ fatty alcohol-aromatic acid ester content in the composition. Most preferably the composition is substantially free of C₁₂-C₁₅ fatty alcohol-aromatic acid esters apart from compound(s) of the first aspect.

In certain embodiments the composition of the second aspect constitutes at least 40% wt, preferably at least 50% wt, more preferably at least 70% wt, most preferably at least 90% wt, of the total weight of all esters of fatty alcohols and acids in the composition. Most preferably the composition is substantially free of all esters of fatty alcohols and acids apart from the compound(s) of the first aspect.

The esters of the first aspect have valuable properties. The representative compound of formula II is substantially non-greasy, shows lack of oiliness and greasiness, has very low cloud point and pour point, has a bland odor, low toxicity and is stable. The representative compound of formula II has good tactile properties, spreadability, solubility in/with sunscreen agents, low tackiness (stickiness), low greasiness on the skin, good suspension properties (TiO₂) and is effective in wetting and dispersing pigments. These properties make it useful as vehicle or carrier emollient or solubilizer for toiletry and cosmetic formulations such as sunscreen creams, hair creams, hand creams, cold creams, manual and electric pre-, during- and post-shave compositions, fingernail polish, topical pharmaceutical ointments, lipsticks, skin lotion and creams, as well as other formulations. A particular use for the esters of the first aspect of this invention is as emollients for antiperspirant, deodorant, sun-screening and personal cleansing compositions. The ester of formula I or II, when added to cosmetic compositions, incorporates in the cosmetic composition one or more, or all, of the following features:
Ease of emulsification;
High refractive index;
Emolliency with good after feel;
Lack of greasiness/pleasant skin feel;
Lack of oiliness while imparting good lubrication;
Low cloud point and pour point;
High spreading coefficient;
Alcohol solubility;
Low toxicity;
Hydrolytic stability; and
Solvent for many skin and hair additives including sunscreens.

An ester of the first aspect has properties in addition to those mentioned hereinbefore when used as an emollient in a sunscreen agent. In this regard, it has been found that when ester of the first aspect is incorporated as an emollient in a sunscreen agent, it increases the sun protection factor (“SPF”) of the sunscreen agent in these sunscreen compositions. In this manner, one achieves in a sunscreen composition containing the ester of the first aspect, a sun protecting effect not only due to the sunscreen agent but also due to the ester of the first aspect. Therefore, sunscreen agents of a lower sun protecting factor can be utilized in sunscreen compositions containing an ester of the first aspect and achieve the sunscreen protecting factors of higher protecting sunscreen agents.

For best results, as ester of the first aspect should be added to a composition when it is substantially pure and substantially free of other esters of aromatic acids with fatty alcohols. In this manner, it is believed that best results are achieved. However, this should not preclude the use of other fatty acid esters or fatty acids in these compositions, suitably within the percentage limits mentioned above, since the use of the novel ester may still provide enhanced results to cosmetic compositions in which it is incorporated.

An ester of the first aspect may be incorporated into cosmetic or skin treating compositions as an emollient in order to impart to these compositions the aforementioned beneficial properties of this invention. The ester of the first aspect can be used as an ingredient for any topical cosmetic compositions including skin, nail, lip, hair topical compositions not only as an emollient but as a diluent, solvent and liquid carrier. As a solvent, an ester of the first aspect is an effective solubilizer for the cosmetically active agents used in these cosmetic compositions.

In accordance with this invention, an ester of the first aspect of this invention is suitably incorporated in a cosmetic composition in an emollient effective amount. In this manner, the ester is provided in an amount at least sufficient to provide the emollient properties to the cosmetic composition. Emollients are dermatologically acceptable compositions that tend to lubricate the skin, increase the softness and suppleness of the skin, prevent or relieve dryness of the skin and/or protect the skin. These emollients are typically water emissible, oily or waxy material and in order to provide the improved results of this invention, an ester of the first aspect may be supplied as an additive to these cosmetic compositions in an amount which is at least sufficient to provide or improve the emollient properties to the cosmetic composition. In accordance with this invention an ester of the first aspect can suitably be added to the cosmetic composition in any amount which is at least sufficient to impart emolliency to the cosmetic composition. The amount of this mixture used in these cosmetic compositions depends on the type and quantity of other ingredients used and in the amount and type of functional additives that are utilized. Generally the amount of the ester of this invention that is incorporated into these cosmetic compositions ranges from about 0.1% to about 30% by weight, for example about 0.3% to about 20% by weight, based upon the total weight of the cosmetic composition, with amounts from about 5% to about 15% by weight based upon the weight of the composition being especially preferred.

In a third aspect of the present invention there is provided a method of preparing a cosmetic composition, wherein an ester of the first aspect of this invention mixed with other ingredients so as to produce a cosmetic composition of the second aspect of this invention.

An ester of the first aspect can be utilized to produce the beneficial properties set forth above in any conventional topical cosmetic composition such sunscreen creams, hair creams, hand ointments, lipsticks, skin lotions and creams as well as other formulations. The term cosmetic compositions include both cosmetic, skin care as well as lip, hair and nail compositions which are topically applied to the humans to achieve a beneficial action on the surface to which it is applied.

An ester of the first aspect is advantageous in that it may be one or more of non-oily, tasteless, inert, essentially nontoxic and non sensitizing, and stable. The ester of the first aspect may provide one or more of emollient, solubilizer, moisturizer, plasticizer, sunscreen vehicle/solvent, de-oiler/degreaser, and emulsifier/co-emulsifier properties. An ester of the first aspect may possess unusual physico-chemical properties, in particular, excellent spreading coefficient which can make it beneficial and unique component of sophisticated delivery system such as in hand, face, and body creams, lotions, soaps and sticks.

Further an ester of the first aspect may be a solvent and/or vehicle for solid organic ultraviolet (UV) absorbers. It may have a function as a plasticizer for polymers contained in skin care composition, and acts as auxiliary suspending agents capable of assisting in the suspension of ingredients in skincare composition and also may function as a dye leveling agent and dye carrier. Thus, an ester of the first aspect when used in a skin care composition may serve not only as an emollient, but also as a carrier; and it may also exhibit these other functions.

In accordance with this invention, the cosmetic composition whose properties may be enhanced by the addition of the ester of the first aspect of the invention can be any conventional cosmetically active material which provides a therapeutic or beneficial cosmetic action to the human body particularly the skin, lips hair or nails. These cosmetic compositions can be in the form of a cream, lotion, ointment, spray, soap-bar, stick etc. which contain a cosmetically active material. In accordance with this invention the term cosmetically active materials may include anti-aging ingredients, anti-wrinkling agents, minerals, preservatives, antioxidants, moisturizers, vitamins, sunscreen agents, and anti-acne agents, deodorants, antiperspirants etc. Among the preferred cosmetically active material are vitamins such as Vitamin A, B₁, B₂, B₃, B₅, B₆, B₁₂, biotin, Vitamin C, Vitamin D, Vitamin E, folic acid and Vitamin K. Other preferred cosmetically active ingredients which can be utilized in the cosmetic composition of this invention anti wrinkling agents, anti-acne agents, moisturizers such as Vitamin E, mineral oils, diisopropyl adipate.

If the cosmetic composition of the second aspect is in the form of a liquid, it may contain a liquid aqueous carrier medium for the cosmetic composition. The liquid aqueous carrier medium can be water or it can be an oil in water or oil in water liquid emulsion. In accordance with this invention, any cosmetically acceptable carrier which is compatible with the water can be combined with water to form the aqueous carrier medium of the second component system. The aqueous carrier medium can contain one or more oil components. The oil component may be any pharmaceutically or cosmetically acceptable oily material which is substantially insoluble in water. These materials can be found for example in the CTFA International Dictionary of Cosmetic Ingredients as well as the U.S. Pharmacopoeia or other equivalent sources. Suitable oil components include, but are not limited to, natural oils, such as coconut oil; hydrocarbons, such as mineral oil and hydrogenated polyisobutene; sterol derivatives, such as lanolin; animal waxes, such as beeswax, plant waxes, such as carnauba; mineral waxes, such as ozokerite; petroleum waxes, such as paraffin wax; synthetic waxes, such as polyethylene; and mixtures thereof. If the cosmetic composition is in the form of a solid, such as a deodorant stick, bar or soap, it generally contains a solid carrier medium in the cosmetic composition. Any conventional solid carrier medium used to formulate such solid cosmetic compositions can be used in accordance with this invention.

The cosmetic composition of the second aspect may contain in addition stable ingredients which include various antioxidants, stabilizers, moisturizers, preservatives and emulsifiers such as those mentioned hereinbefore. Any conventional antioxidant, moisturizer, stabilizer, preservative, or emulsifier which is stable in the carrier medium can be present.

To the cosmetic compositions of the second aspect, there may be added known conventional cosmetic additives, adjuvants and vehicle substrates. Emulsifiers such as glycerol stearate or methyl glucose sesquistearate may also be present Also present may be organic solvents, such as lower aliphatic alcohols from 1 to 6 carbon atoms, such as ethanol isopropanol or propanol, or glycols such as glycerin or 1,2-propylene glycol. The following can also be included in the final cosmetic product: perfume oils; opacifiers such as ethylene glycol distearate; wetting agents or emulsifiers; anti-bacterial and fungicidal ingredients; thickeners (such as bentonite); pH buffer substances; moisture retaining agents; fragrances or perfumes; perfume oils; colorants (such as natural or synthetic direct dyes but also tinting agents such as fluorescein sodium salt); sunscreens or UV filters; preservatives; antioxidants (such as tocopherols); pyrogenic silicic acid; complexing agents; and also physiologically tolerable inorganic or organic acids, such as phosphoric acid, acetic acid, formic acid, glyoxylic acid, lactic acid, tartaric acid or citric acid; bases; salts (such as sodium chloride); buffers (such as sodium citrate or sodium phosphate); consistency-lending agents; and natural, modified, partly or entirely synthetic polymers (such as chitosan, FMOC chitosan and PVP). Naturally, one skilled in the art will be familiar with which of the various individual adjuvants and vehicle substances to be added in order to obtain a desired formulation of the composition of this invention. However, in accordance with a preferred aspect of this invention, these composition preferably do not contain fatty alcohol esters, other than those of the first aspect of this invention; or if present such further esters are present within limits set out hereinabove.

In accordance with a fourth aspect of the present invention there is provided a method of treating the skin or hair of a person using an ester of an optionally substituted aromatic acid and a branched C₁₃ primary alcohol.

In accordance with a fifth aspect of the present invention there is provided the use of an ester of an optionally substituted aromatic acid and a branched C₁₃ primary alcohol for any purpose or to achieve any benefit stated herein; and particularly but not exclusively for providing one or more of improved sunscreening, improved moisturizing and improved emolliency.

Preferred features of the first and second aspects are also preferred features of the third, fourth or fifth aspects.

This invention is further illustrated by the following Examples.

In the Examples Additive A is a C₁₃ benzoate ester prepared by esterifying 1 mole of isotridecane-1-ol (Marlipal O13) with 1 mole of benzoic acid (i.e. unsubstituted) under standard esterification conditions. The resulting product had an acid value below 0.1 mg KOH/g and had been filtered through a filter press with Whatman Paper No. 4. Additive A was an odourless liquid with a freezing point of −25° C., refractive index of 1.4850 and specific gravity of 0.903 at 25° C.

In the Examples, the chemical names of the trade or generic names set forth in the Examples are illustrated in Table I as follows:

TABLE I
Identification Of Trade Or Generic Listed Preparation Ingredients

| Trade or Generic | | |
|---|---|---|
| Material | Identification | Source |
| Marlipal O13 | Isotridecane-1-ol | Sasol North America |
| alcohol | | Inc. |
| Aminol HCA | Cocoamide dimethicone | Innospec Performance |
| | PEG/PPG-20/30 | Chemicals |
| Finsolv SLB-101 | Hydrophilic alkoxylated | Innospec Performance |
| | benzoate ester | Chemicals |
| Finsolv SLB-201 | Dimethicone PEG-8 | Innospec Performance |
| | benzoate | Chemicals |
| Finsolv TN | C12-C15 alkyl benzoate | Innospec Performance |
| | | Chemicals |
| Finester EH 25 | C12-C15 alkyl octanoate | Innospec Performance |
| | | Chemicals |
| Finester LP | Di C12-C15 alkyl maleate | Innospec Performance |
| | | Chemicals |
| Finsolv PL-62 | Poloxamer 182 dibenzoate | Innospec Performance |
| | | Chemicals |
| Finester DOM-R | Dioctyl maleate | Innospec Performance |
| | | Chemicals |
| Finsolv EMG.20 | Methyl gluceth - 20 | Innospec Performance |
| | benzoate | Chemicals |
| Finsolv PL-355 | Poloxamer 105 benzoate | Innospec Performance |
| | | Chemicals |
| Finsolv BCO-115 | Castor oil benzoate | Innospec Performance |
| | | Chemicals |
| Finsolv BCR-111 | Cetyl ricinoleate | Innospec Performance |
| | | Chemicals |
| Finsolv BOHS-111 | Ethylhexyl hydroxy | Innospec Performance |
| | stearate benzoate | Chemicals |
| Crothix | PEG-150 pentaerythritol | Innospec Performance |
| | tetrastearate | Chemicals |
| Tauranol I 78 C | Sodium cocyl isethionate | Innospec Performance |
| | | Chemicals |
| Tauranol WS | Sodium methyl cocyl | Innospec Performance |
| (conc) | taurate | Chemicals |
| Surfine - AZI-A | Nonoxynol-10 carboxylic | Innospec Performance |
| | acid | Chemicals |
| Natrlfine 137-T | Behenyl benzoate, titanium | Innospec Performance |
| | dioxide | Chemicals |
| Solulan 16 | Laureth-16, Ceteth-16, | Amerchol Edison |
| | Oleth 16 and Steareth-16 | |
| Drakeol 9 | Light mineral oil | Panorco |
| Hystrene 9718 | Stearic acid ethylene | Witco Corporation |
| | diamine | |
| Hampine Na 4 | Tetraacetic acid, sodium | Hampshire Chemical |
| | salt | Corp. |
| Polyglycol E400 | Polyethylene glycol 400 | D.V.C. Limited Inc. |
| Sodium stearate | Sodium stearate | Witco Corporation |
| C7 | | |
| Dow Corning Fluid | Cyclomethicone | Dow Corning |
| 344 | | |
| Dow Corning Fluid | Dimethicone | Dow Corning |
| 200 | | |
| Carbomer | Carbopol ETD 2001 resin | B. F. Goodrich |
| Brij 78 | Steareth-20 | ICI |
| Triclosan | Igason DP 300 | Ciba Geigy |
| Dow Corning Fluid | Cyclomethicone | Dow Corning |
| 345 | | |
| Adol 62 | Stearyl alcohol | Witco Corporation |
| Castor Wax MP 70 | Hydrogenated castor oil | Cas Chemical |
| Reach AZP 908 | Aluminum zirconium | Reheis Inc. |
| | tetrachloro-glycine | |
| | complex | |
| Silica | Cabosil M-5 | Cabot Corp. |
| Germaben II | Diazolidinyl urea | ISP (NJ, USA) |
| Escalol 567 | Benzophenone-3 | ISP |
| Escalol 587 | Octylmethoxycinnamate | ISP |
| Witconol 2314 | Isopropyl myristate | Witco Corporation |
| Witconol 2316 | Isopropyl palmitate | Witco Corporation |
| Finsolv SB | Isostearyl benzoate | Innospec Performance |
| | | Chemicals |
| Finsolv PG22 | Dipropylene glycol | Innospec Performance |
| | dibenzoate | Chemicals |
| Finsolv BOD | Octyldodecyl benzoate | Innospec Performance |
| | | Chemicals |

### EXAMPLE 1

### Solubility and Compatibility of Additive A

The solubility characteristics of Additive A are tabulated below. It is soluble at normal handling and use temperatures in most commonly used solvents, emollients and vehicles employed in cosmetic product formulations.

| | |
|---|---|
| Water | − |
| Ethanol | + |
| Isopropanol | + |
| Mineral Oil | − |
| Glycerin | − |
| Propylene glycol | − |
| Dimethicone copolyol | + |
| (Silwet 7604, 7230) | |
| Cyclomethicone | − |
| (Dow Corning Fluid 244) | |
| Dimethicone | − |
| (Dow Corning fluid 200) | |
| Isopropyl myristate | + |
| Isopropyl palmitate | + |
| Finsolv SLB101 | + |
| Finsolv SLN 201 | + |
| Finsolv PL 62 | + |
| Finsolv PL 355 | + |
| Finsolv BCO 115 | + |
| Finsolv BCR 111 | + |
| Finsolv BOHS 111 | + |
| Finsolv TN | + |
| Finsolv TPP | + |
| Finsolv EMG 20 | + |
| Finsolv BOD | + |
| Finsolv SB | + |
| Finsolv PG22 | + |
| Finester EH 25 | + |
| Finester DOM-R | + |

Key: “+” indicates soluble at ambient temperatures
“−” indicates insoluble at ambient temperatures

### EXAMPLE 2

### Sunscreen Solubilities in Esters (25° C.)

The two most commonly used solid organic crystalline sunscreens are 2 hydroxy-4-methoxybenzophenone (called Benzophenone-3) and Parsol 1789 (butyl-methoxy dibenzoyl methane). These two sunscreens are difficult to dissolve and keep in solution for use in sunscreen formulations for optimal SPF (Sun-Protection Factors). Higher solvency for a sunscreen ingredient is described as it allows higher concentrations of the sunscreen active ingredient in a formulation. This advantageously raises the SPF ratings for the formulations. The liquid organic sunscreens that are commonly used as octylsalicylate (OS) and octyl-methoxycinnamate (OMC). Esters of this invention exhibit superiority over commonly used and marketed cosmetic emollients/materials.

| | Solubility in | Solubility in | Solubility in |
|---|---|---|---|
| | Additive A | Finsolv TN | Finester LP |
| Sunscreen | (%) | (%) | (%) |
| Benzophenone-3 | 25 | 15 | 10 |
| Parsol 1789 | 23 | 13 | 6 |

The high solvency exhibited by the Additive A for the solid crystalline organic sunscreens is an advantageous effect in formulating sunscreen products for the skin care markets. Thus, besides being cosmetic emollients, it is excellent solvent for the above-mentioned sunscreens.

A further aspect of this ester, besides being solubilizers for the sunscreens, renders antiwashoff effects. This effect is very attractive in formulating long lasting sunscreen products allowing the sunscreen to remain on the skin for a longer duration.

### EXAMPLE 3

### Sunscreen Cream

A sunscreen cream was prepared from the following ingredients with the amounts set forth in the table being percent by weight:

| Ingredient/Trade | | | | | | |
|---|---|---|---|---|---|---|
| name | A | B | C | D | E | F |
| I. | 62 | 62 | 62 | 62 | 62 | 62 |
| Water | | | | | | |
| Polyglycol E-400 | 5 | 5 | 5 | 5 | 5 | 5 |
| II. | 7 | — | — | — | — | — |
| Additive A | | | | | | |
| Finsolv TN | — | 7 | — | — | — | — |
| Finester EH-25 | — | — | 7 | — | — | — |
| Finester LP | — | — | — | 7 | — | — |
| Witconol 2314 | — | — | — | — | 7 | — |
| Finester DOM-R | — | — | — | — | — | 7 |
| Finsolv EMG-20 | 6 | 6 | 6 | 6 | 6 | 6 |
| Parsol MCX | 8 | 8 | 8 | 8 | 8 | 8 |
| Escalol 567 | 3 | 3 | 3 | 3 | 3 | 3 |
| Escalol 587 | 5 | 5 | 5 | 5 | 5 | 5 |
| Crothix | 1 | 1 | 1 | 1 | 1 | 1 |
| Cetal | 1 | 1 | 1 | 1 | 1 | 1 |
| Cerasynt SD | 1 | 1 | 1 | 1 | 1 | 1 |
| III. | 1 | 1 | 1 | 1 | 1 | 1 |
| Germaben | | | | | | |

The sunscreen cream was prepared from the ingredients set forth above by the following procedure:
1. Charge the ingredients of Part I starting with water.
2. Bring the temperature to 70° C. to 75° C.
3. Mix well until uniform.
4. Heat ingredients of Part II to 75° C.
5. Add ingredients of Part II to Part I with mixing.
6. Cool to 35° C. with gentle mixing.
7. Add ingredient of Part III.
8. Mix well and cool to 30° C.

Each product was evaluated against Additive A. The amount of sample used was 0.5 cc product administered by syringe. Each product was evaluated for the following attributes and rated on a scale of 1 to 5, whereas 1 being the best. Each product was timed from moment of application to the end feel and judged on its absorption time. The above products were applied onto the forearm and the opposite hand rubbing into the skin using 4 fingers, evaluating each sample. The forearm was evaluated with warm water and wipe dry before evaluating another set of samples. This procedure and scale is used in the rest of the examples in this application

Formulations A to F so prepared were tested for skin feel, emolliency, slip and spreadibility on a scale of 1 to 5 with 1 representing the best and 5 representing poor. Results are as follows:

| Ingredients/Trade Name | A | B | C | D | E | F |
|---|---|---|---|---|---|---|
| Skin feel | 1 | 3 | 4 | 4 | 5 | 5 |
| Emolliency | 1 | 3 | 3 | 4 | 5 | 5 |
| Slip | 1 | 3 | 4 | 4 | 5 | 5 |
| Spreadability | 1 | 3 | 3 | 4 | 5 | 5 |

### EXAMPLE 4

### Sunscreen Stick

A sunscreen stick was prepared from the following ingredients with the amounts set forth in the table being percent by weight:

| Ingredients/Trade Name | A | B | C | D | E | F |
|---|---|---|---|---|---|---|
| Witconol APM | 44 | 44 | 44 | 44 | 44 | 44 |
| Additive A | 25 | — | — | — | — | — |
| Finsolv TN | — | 25 | — | — | — | — |
| Finester EH-25 | — | — | 25 | — | — | — |
| Finester LP | — | — | — | 25 | — | — |
| Witconol 2314 | — | — | — | — | 25 | — |
| Finester DOM-R | — | — | — | — | — | 25 |
| Parsol MCX | 7.5 | 7.5 | 7.5 | 7.5 | 7.5 | 7.5 |
| Escalol 587 | 5.5 | 5.5 | 5.5 | 5.5 | 5.5 | 5.5 |
| Sodium stearate C7 | 8 | 8 | 8 | 8 | 8 | 8 |
| Aminol HCA | 7 | 7 | 7 | 7 | 7 | 7 |
| Water | 3 | 3 | 3 | 3 | 3 | 3 |

The sunscreen stick was prepared from the ingredients set forth above by the following procedure:
1. Charge the ingredients in descending order.
2. Bring the temperature to 80° C.
3. Mix well until uniform.
4. Cool to 60° C.
5. Pour into appropriate molds at 60° C.

Formulations A to F so prepared were tested for slip, spreadibility, skin feel, emolliency, stickiness and water rinse off resistance on a scale of 1 to 5 with 1 representing the best and 5 representing poor. Results are as follows:

| Ingredients/Trade Name | A | B | C | D | E | F |
|---|---|---|---|---|---|---|
| Slip | 1 | 2 | 3 | 4 | 5 | 5 |
| Spreadability | 1 | 3 | 3 | 4 | 5 | 4 |
| Skin feel | 1 | 3 | 3 | 4 | 5 | 5 |
| Emolliency | 1 | 3 | 4 | 4 | 5 | 5 |
| Stickiness | 1 | 3 | 4 | 4 | 5 | 4 |
| Water rinse-off resistance | 1 | 3 | 4 | 4 | 5 | 5 |

### EXAMPLE 5

### Clear Sunscreen Oil

A clear sunscreen oil was prepared from the following ingredients with the amounts set forth in the table being percent by weight:

| Ingredients/Trade Name | A | B | C | D | E | F |
|---|---|---|---|---|---|---|
| I. | 60 | 60 | 60 | 60 | 60 | 60 |
| Dow Corning fluid 344 | | | | | | |
| Dow Corning fluid 200 | 10 | 10 | 10 | 10 | 10 | 10 |
| II. | 10 | — | — | — | — | — |
| Additive A | | | | | | |
| Finsolv TN | — | 10 | — | — | — | — |
| Finester EH-25 | — | — | 10 | — | — | — |
| Finester LP | — | — | — | 10 | — | — |
| Witconol 2314 | — | — | — | — | 10 | — |
| Finester DOM-R | — | — | — | — | — | 10 |
| Parsol MCX | 8 | 8 | 8 | 8 | 8 | 8 |
| Escalol 567 | 4 | 4 | 4 | 4 | 4 | 4 |
| Escalol 587 | 8 | 8 | 8 | 8 | 8 | 8 |

The clear sunscreen oil was prepared from the ingredients set forth above by the following procedure:
1. Mix the ingredients of Part I & II separately, until uniform.
2. When uniform added II to I and stir at 25° C.

All formulations are clear liquid at 25° C. Formulation A to F so prepared were tested for slip, water rinse off resistance, stickiness, feel and emolliency. On a scale of 1 to 5 with 1 representing the best and 5 representing poor. Results are as follows:

| Ingredients/Trade Name | A | B | C | D | E | F |
|---|---|---|---|---|---|---|
| Slip | 1 | 2 | 3 | 4 | 5 | 5 |
| Water rinse-off resistance | 1 | 3 | 3 | 4 | 5 | 4 |
| Stickiness | 1 | 3 | 3 | 4 | 5 | 4 |
| Feel | 1 | 2 | 3 | 4 | 5 | 5 |
| Emolliency | 1 | 2 | 3 | 4 | 5 | 4 |

### EXAMPLE 6

### Moisturizing Hand and Body Lotion

A moisturizing hand and body lotion was prepared from the following ingredients with the amounts set forth in the table being percent by weight:

| Ingredients/Trade Name | A | B | C | D | E | F |
|---|---|---|---|---|---|---|
| I. | 85.95 | 85.95 | 85.95 | 85.95 | 85.95 | 85.95 |
| Water | | | | | | |
| Carbomer | 0.15 | 0.15 | 0.15 | 0.15 | 0.15 | 0.15 |
| Methylparaben | 0.1 | 0.1 | 0.1 | 0.1 | 0.1 | 0.1 |
| Propylparaben | 0.1 | 0.1 | 0.1 | 0.1 | 0.1 | 0.1 |
| Sorbitol 70 | 2 | 2 | 2 | 2 | 2 | 2 |
| Hampine Na | 0.2 | 0.2 | 0.2 | 0.2 | 0.2 | 0.2 |
| Triethanolamine | 0.9 | 0.9 | 0.9 | 0.9 | 0.9 | 0.9 |
| II. | 5 | — | — | — | — | — |
| Additive A | | | | | | |
| Finsolv TN | — | 5 | — | — | — | — |
| Finester EH-25 | — | — | 5 | — | — | — |
| Finester LP | — | — | — | 5 | — | — |
| Witconol 2314 | — | — | — | — | 5 | — |
| Finester DOM-R | — | — | — | — | — | 5 |
| Dracol 9 | 1.6 | 1.6 | 1.6 | 1.6 | 1.6 | 1.6 |
| Cetal | 0.7 | 0.7 | 0.7 | 0.7 | 0.7 | 0.7 |
| Hysterene 9718 | 1.5 | 1.5 | 1.5 | 1.5 | 1.5 | 1.5 |
| Cerysynt SD | 0.8 | 0.8 | 0.8 | 0.8 | 0.8 | 0.8 |
| Promulgen G | 1 | 1 | 1 | 1 | 1 | 1 |

The moisturizing hand and body lotion was prepared from the ingredients set forth above by the following procedure:
1. Charge water and disperse Carbomer in it.
2. Charge balance of Part I ingredients in order each to dissolve.
3. After addition of triethanolamine, heat to 65° C.
4. Mix Part I ingredients together and heat to 60° C.
5. Add Part II ingredients to Part I ingredients with good mixing to form emulsion.
6. Continue mixing while cooling to 25° C. All formulations are opaque flowable lotion with pH 6.5.

Formulations A to F so prepared were tested for skin feel, slip, stickiness, long lasting moisturizing effect and emolliency using a scale of 1 to 5 with 1 representing the best and 5 representing poor. Results are as follows:

| Ingredients/Trade Name | A | B | C | D | E | F |
|---|---|---|---|---|---|---|
| Skin | 1 | 2 | 3 | 4 | 5 | 5 |
| Slip | 1 | 2 | 3 | 4 | 5 | 5 |
| Stickiness | 1 | 2 | 3 | 4 | 5 | 4 |
| Moisturizing effect | 1 | 2 | 3 | 3 | 5 | 5 |
| Emolliency | 1 | 2 | 3 | 4 | 5 | 4 |

### EXAMPLE 7

### Elegant Skin Crème

A elegant skin crème was prepared from the following ingredients with the amounts set forth in the table being percent by weight:

| Ingredients/Trade Name | A | B | C | D | E | F |
|---|---|---|---|---|---|---|
| I. | 75.4 | 75.4 | 75.4 | 75.4 | 75.4 | 75.4 |
| Water | | | | | | |
| Quaternium 89 | 1.2 | 1.2 | 1.2 | 1.2 | 1.2 | 1.2 |
| Triethanolamine | 0.2 | 0.2 | 0.2 | 0.2 | 0.2 | 0.2 |
| Glucamate SSE-20 | 1.8 | 1.8 | 1.8 | 1.8 | 1.8 | 1.8 |
| Solulan 16 | 5 | 5 | 5 | 5 | 5 | 5 |
| II. | 1 | 1 | 1 | 1 | 1 | 1 |
| Cerasynt SD | | | | | | |
| Glucate SS | 0.8 | 0.8 | 0.8 | 0.8 | 0.8 | 0.8 |
| Promulgen G | 2.6 | 2.6 | 2.6 | 2.6 | 2.6 | 2.6 |
| Sodium stearate C7 | 3 | 3 | 3 | 3 | 3 | 3 |
| Additive A | 9 | — | — | — | — | — |
| Finsolv TN | — | 9 | — | — | — | — |
| Finester EH-25 | — | — | 9 | — | — | — |
| Finester LP | — | — | — | 9 | — | — |
| Witconol 2314 | — | — | — | — | 9 | — |
| Finester DOM-R | — | — | — | — | — | 9 |

The skin crème was prepared from the ingredients set forth above by the following procedure:
1. Charge the ingredients of Part I starting with water.
2. Bring the temperature to 70° C. to 75° C.
3. Mix well until uniform.
4. Heat ingredients of Part II to 75° C.
5. Add ingredients of Part II to Part I with mixing.
6. Cool to 25° C. with gentle mixing.

All formulations are soft in appearance and are of pH 6.6. Formulations A to F so prepared were tested for skin feel, slip, stickiness, emolliency, on a scale of 1 to 5 with 1 representing the best and 5 representing poor. The results are as follows:

| Ingredients/Trade Name | A | B | C | D | E | F |
|---|---|---|---|---|---|---|
| Skin feel | 1 | 2 | 3 | 4 | 5 | 5 |
| Slip | 1 | 2 | 3 | 4 | 5 | 5 |
| Stickiness | 1 | 2 | 3 | 4 | 5 | 5 |
| Emolliency | 1 | 2 | 3 | 4 | 5 | 5 |

### EXAMPLE 8

### Deodorant Stick

A deodorant stick was prepared from the following ingredients with the amounts set forth in the table being percent by weight:

| Ingredients/Trade Name | A | B | C | D | E | F |
|---|---|---|---|---|---|---|
| Propylene glycol | 65 | 65 | 65 | 65 | 65 | 65 |
| Water | 15 | 15 | 15 | 15 | 15 | 15 |
| Sodium stearate C7 | 8 | 8 | 8 | 8 | 8 | 8 |
| Brij 78 | 8 | 8 | 8 | 8 | 8 | 8 |
| Additive A | 3.8 | — | — | — | — | — |
| Finsolv TN | — | 3.8 | — | — | — | — |
| Finester EH25 | — | — | 3.8 | — | — | — |
| Finester LP | — | — | — | 3.8 | — | — |
| Witconol 2314 | — | — | — | — | 3.8 | — |
| Finester DOM-R | — | — | — | — | — | 3.8 |
| Triclosan | 0.2 | 0.2 | 0.2 | 0.2 | 0.2 | 0.2 |

The deodorant stick was prepared from the ingredients set forth above by the following procedure:
1. Change the ingredients in descending sequence starting with propylene glycol.
2. Bring the temperature to 80° C. allowing all to dissolve.
3. Mix well until uniform.
4. Cool to 60° C. and cast into stick molds.

Formulations A to F so prepared were tested for emolliency, slip, soft dry and afterfeel using a scale of 1 to 5 with 1 representing the best and 5 representing poor. Results as follows:

| Ingredients/Trade Name | A | B | C | D | E | F |
|---|---|---|---|---|---|---|
| Emolliency | 1 | 2 | 3 | 3 | 5 | 4 |
| Slip | 1 | 2 | 2 | 3 | 5 | 4 |
| Soft dry after-feel | 1 | 2 | 2 | 3 | 5 | 4 |

### EXAMPLE 9

### Non-Whitening Antiperspirant Stick

A non-whitening antiperspirant stick was prepared from the following ingredients with the amounts set forth in the table being percent by weight:

| Ingredients/Trade Name | A | B | C | D | E | F |
|---|---|---|---|---|---|---|
| I. | 39 | 39 | 39 | 39 | 39 | 39 |
| Dow Corning 345 Fluid | | | | | | |
| Adol 62 | 18 | 18 | 18 | 18 | 18 | 18 |
| Castorwax MP-70 | 5 | 5 | 5 | 5 | 5 | 5 |
| Additive A | 5 | — | — | — | — | — |
| Finsolv TN | — | 5 | — | — | — | — |
| Finester EH 25 | — | — | 5 | — | — | — |
| Finester LP | — | — | — | 5 | — | — |
| Witconal 2314 | — | — | — | — | 5 | — |
| Finester DOM-R | — | — | — | — | — | 5 |
| Finsolv 116 | 10 | 10 | 10 | 10 | 10 | 10 |
| II. | 20 | 20 | 20 | 20 | 20 | 20 |
| Reach AZP-908 | | | | | | |
| Talc | 2 | 2 | 2 | 2 | 2 | 2 |
| Silica | 1 | 1 | 1 | 1 | 1 | 1 |

The non-whitening antiperspirant stick was prepared from the ingredients set forth above by the following procedure:
1. Charge the ingredients of Part I starting with Dow Corning Fluid 345.
2. Bring the temperature to 75° C.
3. Mix well until uniform.
4. Add Part II powders, mix until completely dispersed. Maintain 75° C.
5. Cool to 55° C. and cast in to stick molds.

Formulation A to F so prepared were tested for emolliency, stickiness, talc like feel, and stick structure. On a scale of 1 to 5 with 1 representing the best and 5 representing poor. The results are as follows:

| Ingredients/Trade Name | A | B | C | D | E | F |
|---|---|---|---|---|---|---|
| Emolliency | 1 | 2 | 3 | 3 | 5 | 4 |
| Stickiness | 1 | 2 | 2 | 3 | 5 | 5 |
| Talc-like feel | 1 | 2 | 2 | 3 | 5 | 4 |
| Stick structure | 1 | 2 | 3 | 3 | 5 | 5 |

### General Comment Applicable Throughout

In this specification, including in the examples, percentage values are expressed in terms of weight by total composition weight (wt/wt) unless otherwise stated.

1. An ester of an optionally substituted aromatic acid and a branched C₁₃ primary alcohol.

2. An ester as claimed in claim 1 wherein the optionally substituted aromatic acid is optionally substituted benzoic acid.

3. An ester as claimed in claim 1 wherein the aromatic acid is unsubstituted.

4. An ester as claimed in claim 1 wherein the branched C₁₃ primary alcohol residue is branched from one position only, on the backbone of the primary alcohol.

5. An ester as claimed in claim 1 wherein the branched C₁₃ primary alcohol residue contains from 1 to 6 carbon atoms, preferably a methyl group, in one or more side chains.

6. An ester as claimed in claim 1 wherein the branched C₁₃ primary alcohol residue is branched from the penultimate carbon atom in the backbone, with reference to the optionally substituted aromatic moiety.

7. An ester as claimed in claim 1 having the formula

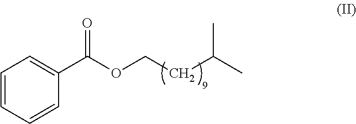

8. An ester as claimed in claim 1 provided in at least 90% by weight pure form, preferably at least 95%, preferably at least 98%, preferably substantially pure.

9. A composition for application to the person, the composition comprising as one component an ester as claimed in claim 1.

10. A composition as claimed in claim 9 comprising a carrier or diluent or solvent; and/or a cosmetically effective ingredient, for example a sunscreen agent, and a deodorant agent or an antiperspirant agent.

11. A composition as claimed in claim 9 wherein said ester constitutes at least 40% wt, preferably at least 50% wt, more preferably at least 70% wt, most preferably at least 90% wt, and preferably substantially all, of the total weight of C₁₂-C₁₅ fatty alcohol-aromatic acid ester content in the composition.

12. A composition as claimed in claim 9 wherein said ester constitutes at least 40% wt, preferably at least 50% wt, more preferably at least 70% wt, most preferably at least 90% wt, and preferably substantially all, of the total weight of all esters of fatty alcohols and acids in the composition.

13. A composition as claimed in claim 9 wherein said ester is present in the amount of from about 0.1% to about 30% by weight, based upon the total weight of the composition.

14. A composition as claimed in claim 9 wherein said ester functions in the composition as an emollient and is the only emollient present in the composition.

15. A composition as claimed in claim 9 wherein the composition is a sunscreen composition and contains a sunscreen agent whose effectiveness is enhanced by said ester.

16. A method of preparing a composition as claimed in claim 9 wherein an ester as claimed in claim 1 is mixed with other ingredients so as to produce said composition.

17. A method of treating the skin or hair of a person comprising administering an ester of an optionally substituted aromatic acid and a branched C₁₃ primary alcohol.

18. (canceled)
